(19) United States
(12) Patent Application Publication
(10) Pub. No.: US 2026/0314568 A1
(43) Pub. Date: Oct. 15, 2026

(54) POULTRY FARMING STRUCTURE, FEED AND METHOD, AND BREEDING METHOD

(71) Applicants: MANUKA (XIAMEN) BIOTECH CO., LTD, Xiamen, Fujian (CN); MANUKA (LONG YAN) BIOTECH CO., LTD, Longyan, Fujian (CN)

(72) Inventors: Zhouwen LAI, Xiamen, Fujian (CN); Guihua DENG, Longyan, Fujian (CN); Liang ZHANG, Xiamen, Fujian (CN); Yeling YU, Xiamen, Fujian (CN); Yizhu HUANG, Xiamen, Fujian (CN); Jinying YANG, Xiamen, Fujian (CN); Yugan YANG, Longyan, Fujian (CN)

(21) Appl. No.: 19/104,494

(22) PCT Filed: Aug. 22, 2023
(86) PCT No.: PCT/CN2023/114288

§ 371 Date: Feb. 18, 2025

(30) Foreign Application Priority Data
Aug. 22, 2022 (CN) 202222207297.1

(51) Int. Cl. A01K 39/01

(52) U.S. Cl. CPC A01K 39/01

(57) ABSTRACT

The present disclosure is a poultry farming structure, feed and method, and a breeding method. The poultry farming structure includes a culture region and breeding regions. A housing of the culture region is transparent or semitransparent, so that light irradiates into the culture region from outside or the culture region is in communication with the outside. The culture region is in communication with the breeding regions. First, the culture region is transparent or semitransparent, so that chlorella in the culture region can better receive external sunlight, thereby accelerating the reproduction of the chlorella. Second, the culture region is in communication with the breeding regions, so that the chlorella located in the culture region can be more conveniently and quickly conveyed into the breeding regions.

### CROSS-REFERENCE TO RELATED APPLICATION

This application claims priority to Chinese Patent Application No. 202222207297.1 filed with the China National Intellectual Property Administration on Aug. 22, 2022, and entitled “POULTRY FARMING STRUCTURE, FEED AND METHOD, AND BREEDING METHOD”, which is incorporated herein by reference in its entirety.

### FIELD

The present application belongs to the field of poultry farming, and in particular to a poultry farming structure, feed and method, and a breeding method.

### BACKGROUND

Algae such as chlorella have been widely used as feed for poultry (such as ducks) at present. However, an existing method requires mixing the chlorella into feed and stirring and then distributing into each poultry breeding region. The method is relatively complex in operation and not conducive to industrialized poultry farming.

### SUMMARY

The present application provides a poultry farming structure to facilitate supplying of algae feed to poultry.

In a first aspect, the present application provides a poultry farming structure, including a culture region and breeding regions; a housing of the culture region is transparent or semitransparent, so that light irradiates into the culture region from outside or the culture region is in communication with the outside, wherein the culture region is in communication with the breeding regions.

In some embodiments, the poultry farming structure includes a plurality of compartments arranged from top to bottom in sequence, and each compartment includes a plurality of breeding regions, wherein the culture region is in communication with the breeding regions of each compartment through a plurality of communicating channels.

In some embodiments, the breeding regions are provided with wading areas, and the communicating channels are connected to the wading areas in the breeding regions. In some embodiments, parts of the communicating channels located in the breeding regions are provided with openings.

In some embodiments, parts of the communicating channels located in the breeding regions are flush with bearing surfaces of the breeding regions or parts of the communicating channels located in the breeding regions are 0-50 cm higher than bearing surfaces of the breeding regions, and the openings are located at tops of the communicating channels, wherein the bearing surfaces are surfaces for bearing poultry.

In some embodiments, parts of the communicating channels located in the breeding regions are more than 100 cm higher than the bearing surfaces of the breeding regions, and bottoms of the communicating channels are provided with spray openings, wherein the bearing surfaces are surfaces for bearing poultry.

In some embodiments, a valve is arranged between the plurality of communicating channels and the culture region.

In some embodiments, the culture region is located at the top of the poultry farming structure.

In some embodiments, the poultry farming structure further includes a liquid pump, and the liquid pump is configured to convey liquid to the culture region.

In a second aspect, the present application provides a poultry farming method, including allowing poultry to ingest chlorella and duckweed.

In some embodiments, the poultry may ingest chlorella and duckweed simultaneously or alternately.

In some embodiments, the two ingredients are provided in a form of a single component, and not necessarily provided simultaneously in timing.

In some embodiments, the two ingredients are mixed together and ingested by poultry.

In some embodiments, the chlorella is ingested by adding into drinking water for poultry.

In some embodiments, the chlorella is ingested by adding into drinking water for poultry, and the concentration of the chlorella in the drinking water for poultry is 6*10⁶/ml, 7*10⁶/ml, 8*10⁶/ml, 9*10⁶/ml, 10*10⁶/ml, or 11*10⁶/ml, 12*10⁶/ml, 13*10⁶/ml, 14*10⁶/ml, 15*10⁶/ml, 16*10⁶/ml, 17*10⁶/ml, 18*10⁶/ml, 19*10⁶/ml, 20*10⁶/ml or more.

In some embodiments, the chlorella is ingested by adding into feed or feed additives for poultry.

In some embodiments, the duckweed is cultured in wading areas for poultry, and poultry ingests the duckweed in the wading areas.

In a third aspect, the present application provides a poultry feed, including chlorella and duckweed.

In some embodiments, the two ingredients are provided in a form of a single component.

In some embodiments, the two ingredients are provided in a form of a composition. In a fourth aspect, the present application provides a method for purifying aquaculture water, including conveying chlorella to the aquaculture water, and culturing duckweed on the surface of the aquaculture water at the same time.

In some embodiments, the concentration of the chlorella conveyed to the aquaculture water is 6*10⁶/ml, 7*10⁶/ml, 8*10⁶/ml, 9*10⁶/ml, 10*10⁶/ml, or 11*10⁶/ml, 12*10⁶/ml, 13*10⁶/ml, 14*10⁶/ml, 15*10⁶/ml, 16*10⁶/ml, 17*10⁶/ml, 18*10⁶/ml, 19*10⁶/ml, 20*10⁶/ml or more.

In some embodiments, the duckweed accounts for at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% of the surface of the aquaculture water.

In a fifth aspect, the present application provides a three-dimensional ecological breeding method, which is applied to the above poultry farming structure, and includes:
adding chlorella to the culture region;
culturing duckweed in the wading areas; and
conveying a liquid containing the chlorella to the wading areas through the communicating channels.

In some embodiments, the concentration of the chlorella in the wading areas is 6*10⁶/ml, 7*10⁶/ml, 8*10⁶/ml, 9*10⁶/ml, 10*10⁶/ml, or 11*10⁶/ml, 12*10⁶/ml, 13*10⁶/ml, 14*10⁶/ml, 15*10⁶/ml, 16*10⁶/ml, 17*10⁶/ml, 18*10⁶/ml, 19*10⁶/ml, 20*10⁶/ml or more.

In some embodiments, water in the wading areas is discharged into a fishpond.

In some embodiments, water in the fishpond is discharged into a settling pond.

In some embodiments, water in the settling pond is discharged into a greenhouse and an orchard.

In some embodiments, the culture region is transparent or semitransparent, so the chlorella in the culture region grows better, thereby accelerating the reproduction of the chlorella. The culture region is in communication with the breeding regions, so that the chlorella located in the culture region may be conveyed into the breeding regions. The culture region is located at the top of the poultry farming structure, so that the chlorella located in the culture region may better receive sunlight and grow.

### BRIEF DESCRIPTION OF THE DRAWINGS

FIG. 1a is a schematic structural diagram of a poultry farming structure according to an embodiment;

FIG. 1b is a schematic structural diagram of a poultry farming structure according to another embodiment;

FIG. 1c is a schematic structural diagram of a poultry farming structure according to another embodiment;

FIG. 1d is a schematic diagram of a front view of a structure of a top of the poultry farming structure according to FIG. 1b;

FIG. 1e is a schematic diagram of a top view of a structure of a top of the poultry farming structure according to FIG. 1b;

FIG. 2 is a schematic structural diagram of a certain compartment according to an embodiment;

FIG. 3 is a side cross-sectional view of a breeding region in a poultry farming structure according to an embodiment;

FIG. 4 is a schematic diagram of a bottom view of a breeding region in a poultry farming structure according to an embodiment;

FIG. 5 is an enlarged view of area A in FIG. 4;

FIG. 6 is a photograph of chlorella fluid after primary culture of chlorella;

FIG. 7 is a photograph of chlorella fluid after secondary culture of chlorella;

FIG. 8 is a photograph of chlorella fluid when testing the secondary culture of chlorella on Apr. 28, 2021;

FIG. 9 is a photograph of chlorella fluid when testing the secondary culture of chlorella on May 6, 2021;

FIG. 10 is a photograph of chlorella fluid when testing the secondary culture of chlorella on May 13, 2021;

FIG. 11 is a photograph of chlorella fluid when testing the secondary culture of chlorella on May 20, 2021; and

FIG. 12 is a schematic structural diagram of a study experiment.

In figures, 1: column; 5: dropping board; 7: wading area; 71: reservoir; 8: transitional area; 9: laying area; 13A and 13B: drinking water pools; 14A and 14B: troughs; 15: top; 100: culture region; 110: culture pipeline; 120: light source device; 130: first inlet; 140: second inlet; 200: breeding region; 300: compartment; 400: longitudinal conveyor pipe; 500: valve. 600: liquid pump; 700: liquid pipeline; 800: communicating channel; 900: bearing surface.
B1: small pen; B11: laying area of a small pen; B12: activity area of a small pen; B13: pool of a small pen; B2: first big pool; B21: laying area of a first big pool; B22: activity area of a first big pool; B23: wading area of a first big pool; B3: second big pool; B31: laying area of a second big pool; B32: activity area of a second big pool; B33: wading area of a second big pool; B4: fishpond; B5: settling pond; B6: greenhouse; B7: orchard; I: water inlet; and O: water outlet.

### DETAILED DESCRIPTION

The technical solutions of the present application are further illustrated by the specified embodiments, which are not intended to limit the scope of the present application. Some non-essential modifications and adaptations made by others in accordance with the concept of the present application remain within the scope of the present application.

The above description is provided with reference to the accompanying drawings to facilitate a comprehensive understanding of the various embodiments of the present application as defined by the claims. It contains various specific details to facilitate such understanding, but these details should be regarded as exemplary only. Accordingly, those of ordinary skill in the art will recognize that changes and improvements may be made to the various embodiments described herein without departing from the scope of the present application as defined by the claims. In addition, descriptions of well-known functions and structures may be omitted for the sake of clarity and brevity.

The terms and words used in the following description and claims are not limited to the written meaning, but are used only by the inventor to allow a clear and consistent understanding of the present application. Accordingly, it is obvious to those of ordinary skill in the art that the following description of the various embodiments of the present application is provided for the purpose of explanation only, and not for the purpose of limiting the present application as defined by the claims.

In the descriptions of the application, it is to be understood that orientation or position relationships indicated by terms “center”, “upper”, “lower”, “front”, “back”, “left”, “right”, “vertical”, “horizontal”, “top”, “bottom”, “inner”, “outer”, and the like are orientation or position relationships shown in the drawings, are adopted not to indicate or imply that indicated devices or components must be in specific orientations or structured and operated in specific orientations but only to conveniently describe the application and simplify descriptions. Thus, it should not be construed as limitations to the present application. In addition, in the descriptions of the present application, “multiple” means two or more than two, unless otherwise specified.

In addition, technical features involved in the different embodiments of the present application described below can be combined with each other as long as they do not conflict with each other.

It should be understood that the singular forms “one”, “a”, “said” and “the” include plural references unless the context clearly stipulates otherwise. In the present application, the expression “or” includes any or all combinations of the words listed together. For example, “A or B” can include A or B, or can include both A and B. It should also be understood that as used herein, the term “and/or” used in the present application refers to and includes any or all possible combinations of one or more associated listed items.

Throughout the specification and claims of the present application, the words “include” and “contain” and variations of the words, such as “including” and “containing” mean “including but not limited to”, and are not intended to (and will not) exclude other parts, wholes or steps.

In the descriptions of the embodiments of the present application, it is to be noted that, unless otherwise definitely specified and limited, terms “arrange”, “provide”, and “connect” should be broadly understood. For example, the terms may refer to fixed connection and may also refer to detachable connection or integrated connection. The terms may refer to mechanical connection and may also refer to electrical connection. The terms may refer to direct connection, may also refer to indirect connection through a medium, and may refer to communication in two components. For those of ordinary skill in the art, specific meanings of these terms in the application can be understood according to a specific condition.

It should be noted that like numbers and letters refer to like items in the following figures, and thus, once an item is defined in one figure, it does not require further definition and explanation in subsequent figures.

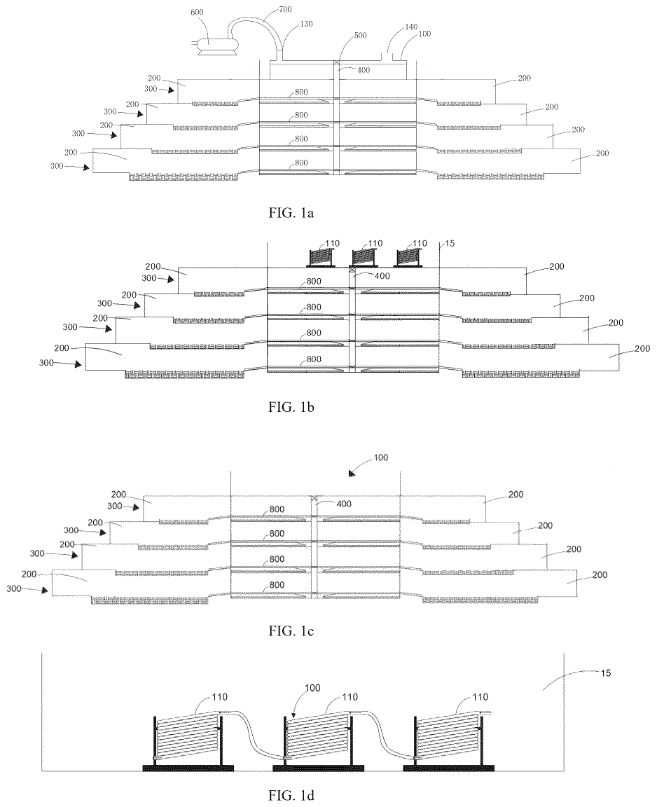

As shown in FIG. 1a, a poultry farming structure includes a culture region 100 and breeding regions 200, and a housing of the culture region 100 is transparent or semitransparent, so that light irradiates into the culture region 100 from outside, wherein the culture region 100 is in communication with the breeding regions 200. The housing of the culture region 100 is configured to be transparent or semitransparent, so that light irradiates into the culture region 100 from outside, in order to culture algae such as chlorella, which may be ingested by poultry, in the culture region 100. Meanwhile, the culture region 100 is in communication with the breeding regions 200, so a user may introduce a liquid to facilitate the movement of the chlorella from the culture region 100 into the breeding regions 200 along with liquid flow.

In some embodiments, the poultry farming structure includes a plurality of compartments 300 arranged from top to bottom in sequence. Each compartment 300 includes a plurality of breeding regions 200, and the culture region 100 is in communication with the breeding regions 200 of each compartment 300 through a plurality of communicating channels 800. Specifically, the culture region 100 is located at the top of the poultry farming structure. Each compartment 300 is equipped with one or a plurality of communicating channels 800, and the culture region 100 is in communication with each communicating channel 800 through a longitudinal conveyor pipe 400 from top to bottom.

In some embodiments, a plurality of valve 500 is arranged between each communicating channel 800 and the culture region 100. More specifically, a valve 500 is arranged at a connection area of the culture region 100 and the longitudinal conveyor pipe 400, and the other valves 500 are arranged at a connection area of the longitudinal conveyor pipe 400 and each communicating channel 800. Through the design of the valve 500, the transport of the chlorella between the culture region 100 and the communicating channels 800 can be controlled, allowing the chlorella in the culture region 100 to be transported to different communicating channels 800 as required.

In some embodiments, only one valve 500 may be arranged between the culture region 100 and the plurality of communicating channels 800, and when a liquid is pumped to the culture region 100 at this moment, chlorella may be conveyed to all communicating channels 800. A design of the valve 500 may depend on specific circumstances, which is not excessively limited in the disclosure.

In some embodiments, a liquid is conveyed to the culture region 100 through a liquid pump 600, so that the chlorella may be conveyed to the communicating channels 800 and the breeding regions 200. More specifically, the culture region 100 is provided with a first inlet 130 and a second inlet 140. The first inlet 130 is in communication with an external water source, water from the external water source is conveyed to the culture region 100 through the liquid pump 600 via a liquid pipeline 700, and the second inlet 140 is used to add chlorella. Poultry farming personnel may add chlorella into the culture region 100 through the second inlet 140. Moreover, in some embodiments, a conveyor belt or a similar structure may be equipped to convey the chlorella into the second inlet 140.

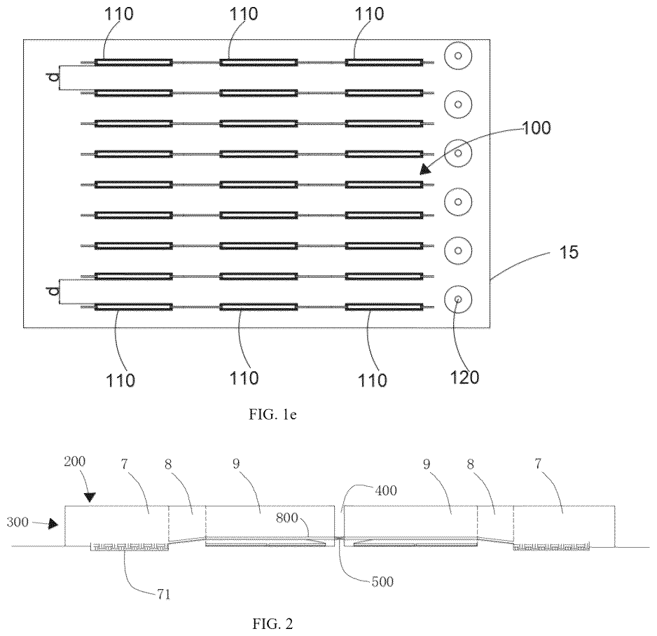

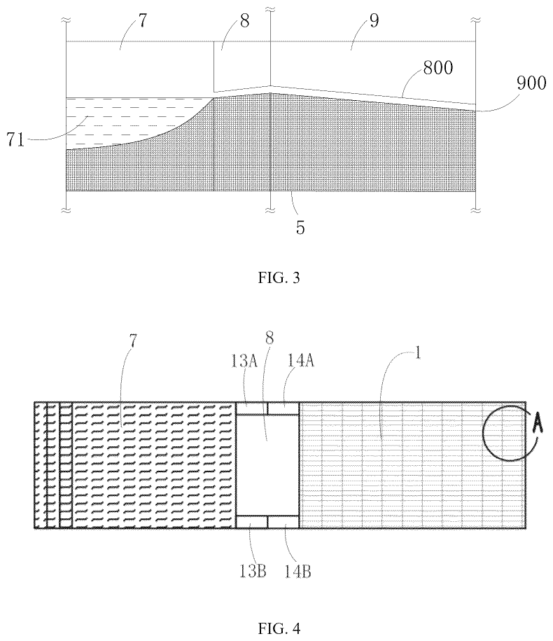

In some embodiments, as shown in FIG. 2 and FIG. 3, the breeding regions 200 are provided with wading areas 7, and the communicating channels 800 are connected to the wading areas 7 in the breeding regions 200. In some embodiments, each breeding region includes a wading area 7, a transitional area 8, and a laying area 9. The wading area 7 is connected with one end of the transitional area 8, and the other end of the transitional area 8 is connected with the laying area 9. The wading area 7 and the transitional area 8 are semi-open environments; the laying area 9 is an indoor environment; the laying area 9 is separated from the transitional area 8 by a wall; and an automatic rolling gate is arranged in the wall. The automatic rolling gate is opened before night falls, and ducks are driven indoors; and the automatic rolling gate automatically descends in the daytime, and the ducks are driven to the wading area 7 and the transitional area 8 to dabble and pick and eat food outdoors. The automatic rolling gate ascends or descends according to species of ducks, production management, laying habit, antiepidemic demand, and season and weather change.

In some embodiments, parts of the communicating channels 800 located in the breeding regions 200 are provided with openings.

In some embodiments, parts of the communicating channels 800 located in the breeding regions 200 are flush with bearing surfaces of the breeding regions 200 or parts of the communicating channels 800 located in the breeding regions 200 are 0-50 cm higher than bearing surfaces 900 of the breeding regions 200, and the openings are located at tops of the communicating channels 800, wherein the bearing surfaces 900 are surfaces for bearing poultry. More specifically, the openings are arranged in laying areas 9 and/or transitional areas 8, so that poultry sucks the chlorella in the communicating channels 800 through the openings. A tail end of each communicating channel 800 is in communication with the wading area 7, the chlorella in the culture region 100 enters the communicating channel 800 from a head end of the communicating channel 800, then leaves the communicating channel 800 from the tail end of the communicating channel 800, and enters the wading area 7. It should be understood that the communicating channels 800 only occupy parts of the laying areas 9 and/or the transitional areas 8, and a large area remains for activity of poultry.

Moreover, in some embodiments, parts of the communicating channels 800 located in the breeding regions 200 are more than 100 cm higher than bearing surfaces 900 of the breeding regions 200, and bottoms of the communicating channels 800 are provided with spray openings. In the above embodiment, a communicating channel 800 located at a relatively high horizontal position is arranged, so as to spray a liquid containing chlorella to poultry located in the lower breeding regions 200. Of course, in some embodiments, parts of the communicating channels 800 located in the breeding regions 200 are more than 150 cm and even more than 200 cm higher than bearing surfaces 900 of the breeding regions 200.

It should be noted that the wading area 7 includes a reservoir 71 where laying ducks can engage in activities and play during the day and part of laying ducks lay eggs. Water in the reservoir 71 is changed every day to keep water fresh and clean. One end of the wading area 7 far from the transitional area 8 is partially open-air, to ensure that ducks receive sunlight.

As shown in FIG. 4, the transitional area 8 allows laying ducks to inhabit and preen in the daytime; and two sides of the transitional area 8 are provided with drinking water pools 13A and 13B and troughs 14A and 14B for the laying ducks to drink water and pick and eat food. The drinking water pools 13A and 13B may supplement water by auto-induction, and the troughs 14A and 14B can achieve automatic feed to ensure precision feeding. When laying ducks go ashore from the reservoir 71, the laying ducks may drip water drops in the transitional area 8, so that the laying ducks may keep rooms dry to the greatest extent when entering the rooms.

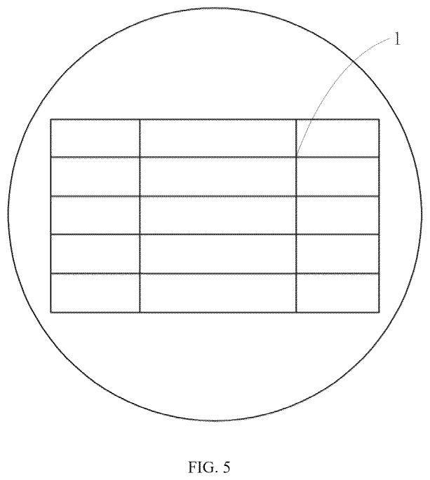

The laying area 9 and the transitional area 8 are connected to serve as indoor environments; and the laying area 9 includes a bearing platform for laying ducks to rest at night and lay eggs. The bearing platform includes a hollow structure, as shown in FIG. 4 and FIG. 5. The hollow structure consists of a plurality of parallel columns 1, specifically, hollow stainless steel tubes and a plurality of cross bars vertically connected with the parallel stainless steel tubes. A plane formed by axes of the plurality of cross bars and a plane formed by axes of the plurality of stainless steel tubes are not the same plane; the cross bars are located under the stainless steel tubes; and each cross bar is connected with lower surfaces of the plurality of stainless steel tubes. A plane formed by upper surfaces of the plurality of parallel stainless steel tubes (i.e. columns 1) forms a part of a bearing surface 900 for bearing poultry, laying ducks can stand thereon, and width of gaps between the stainless steel tubes is required to ensure that feet of ducks do not fall into the gaps and get stuck. Moreover, the cross bars bear and fix the stainless steel tubes. The axes of the stainless steel tubes are vertical to a wall connecting the laying area 9 and the transitional area 8 from a top-down perspective. After laying ducks lay eggs on the bearing surface 900, the eggs may roll to the lowest position of the hollow structure along gaps between the stainless steel tubes. It should be understood that the configuration of the bearing surface 900 is varied, and not limited to the above embodiment. For example, in some embodiments, the bearing surface 900 may be a flush cement floor or a steel plate.

The laying area 9 also includes a dropping board 5. The dropping board 5 is located under the bearing platform and is arranged horizontally, and liquid dung generated by laying ducks in the laying area passes through the hollow structure and finally drops onto the dropping board 5. Furthermore, the dropping board 5 is further provided with an automatic dung scraping device which may automatically clear the liquid dung from the dropping board 5.

It should be understood that in some embodiments, each breeding region 200 includes the wading area 7, the transitional area 8, and the laying area 9, but in other embodiments each breeding region 200 may not include the wading area 7, the transitional area 8, and the laying area 9, or only includes one or two thereof, and specific structures of the wading area 7, the transitional area 8, and the laying area 9 may be adjusted according to circumstances and are not only limited to the above description.

Dimensions of all parts in a laying duck farming mechanism provided by this embodiment are as follows:

The wading area 7 has a length of 2.6 meters, a width of 2 meters, and a height of 1 meter. The transitional area 8 has a length of 1 meter along an oblique direction, and a width of 1 meter equal to that of the wading area 7. The laying area has a length of 3.6 meters, and a width of 1 meter equal to that of the wading area 7.

As shown in FIG. 1a, the culture region 100 is formed by a pipeline. However, in some embodiments, the culture region 100 may be formed by matching a plurality of culture pipelines 110. Specifically, as shown in FIG. 1b and FIG. 1d, the top 15 of the poultry farming structure may be provided with a plurality of culture pipelines 110. The plurality of culture pipelines 110 are converged as required and then in communication with communicating channels 800, or are respectively in communication with the same or different communicating channels 800, which is not specifically limited by the disclosure. Moreover, the roof of the top 15 of the poultry farming structure may be provided with an opening to receive external sunlight, or the roof and/or side faces of the top 15 of the poultry farming structure are of transparent or semitransparent structures, so that sunlight may pass through the roof and/or the side faces to irradiate the top 15 of the poultry farming structure. Meanwhile, to reduce the influence of cloudy or rainy days on light, a light source device 120 is arranged on the top of the poultry farming structure to provide light. It should be noted that to better provide light to chlorella in the culture region 100, the culture region 100 is arranged on the top 15 of the poultry farming structure. However, in some embodiments, the culture region 100 may be arranged at different positions according to circumstances, and the light source device 120 also may be arranged at different positions according to circumstances. For example, the light source device 120 may be arranged outside the poultry farming structure, or on a bottom or a middle layer of the poultry farming structure.

A housing of the culture pipeline 110 is of a transparent or semitransparent structure, so that light may pass through the housing of the culture pipeline 110 and irradiate chlorella therein.

It should be understood that the light source device 120 may be an incandescent lamp, a light-emitting diode (LED) lamp, a red-infrared lamp, etc., which is not specifically limited by the present application.

In some embodiments, the poultry farming structure further includes an oxygen conveying structure, such as an aerator or an air pump (one end of the air pump may be connected with an oxygen bottle or other devices for storing oxygen), and oxygen is conveyed to the culture pipeline 110 through the oxygen conveying structure.

In some embodiments, as shown in FIG. 1e, a plurality of culture pipelines 110 are arrayed to form a plurality of culture pipeline rows, and a distance d exists between each culture pipeline row and an adjacent culture pipeline, and allows people to pass. Specifically, the distance is 30-250 cm.

However, in some embodiments, as shown in FIG. 1c, the culture region 100 is in communication with outside, namely the culture region is of an open structure. Specifically, the culture region 100 is a pool with an opening on the top, and sunlight may irradiate into the culture region 100 through the opening on the top of the culture region 100. A liquid may be added into the culture region 100 to culture chlorella. In some embodiments, a water outlet is arranged in the culture region 100 (more specifically, the water outlet is arranged on a bottom of the culture region 100). The water outlet is in communication with the communicating channels 800 (specifically, the water outlet is in communication with the communicating channels 800 through a longitudinal conveyor pipe), and after leaving the culture region 100 through the water outlet, chlorella goes to different breeding regions 200 through the communicating channels 800.

In addition, some embodiments also provide a method for purifying aquaculture water, including conveying chlorella to the aquaculture water, and culturing duckweed on the surface of the aquaculture water at the same time. The aquaculture water may be the poultry farming structure or other water for poultry farming, which is not specifically limited by this embodiment.

In some embodiments, the concentration of the chlorella conveyed to the aquaculture water is 6*10⁶/ml, 7*10⁶/ml, 8*10⁶/ml, 9*10⁶/ml, 10*10⁶/ml, or 11*10⁶/ml, 12*10⁶/ml, 13*10⁶/ml, 14*10⁶/ml, 15*10⁶/ml, 16*10⁶/ml, 17*10⁶/ml, 18*10⁶/ml, 19*10⁶/ml, 20*10⁶/ml or more. Specific values are not limited.

In some embodiments, the duckweed accounts for at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% of the surface of the aquaculture water.

In addition, some embodiments also provide a method for purifying aquaculture water, which is characterized by conveying chlorella to the aquaculture water, and culturing duckweed on the surface of the aquaculture water at the same time.

In some embodiments, the concentration of the chlorella conveyed to the aquaculture water is 6*10⁶/ml, 7*10⁶/ml, 8*10⁶/ml, 9*10⁶/ml, 10*10⁶/ml, or 11*10⁶/ml, 12*10⁶/ml, 13*10⁶/ml, 14*10⁶/ml, 15*10⁶/ml, 16*10⁶/ml, 17*10⁶/ml, 18*10⁶/ml, 19*10⁶/ml, 20*10⁶/ml or more.

In some embodiments, the duckweed accounts for at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% of the surface of the aquaculture water.

A plurality of studies by the applicant reveal that by providing drinking water containing chlorella to poultry and culturing duckweed on the surface of the wading area for poultry, the drinking water containing chlorella flows into the wading area for poultry, so the chlorella also enters water of the wading area, and degrades feces emitted by poultry into the wading area, to ensure that water quality is difficult to reduce. Meanwhile, the feces emitted by poultry into the wading area may be used as a nutrient for the duckweed, and ingested by the duckweed to help growth, to form a three-dimensional recirculating ecological breeding mode.

The study experiment of the applicant is as follows:

### General Materials and Methods

### 1) Culture and Use of Chlorella in a Duck Farm

### Operating Steps

1. Preparation of apparatus and raw and auxiliary materials
1.1 Two 5-ton culturing vats and a 0.75 KW circulating pump were prepared and connected in series.
1.2 Various ingredients were weighed according to instructions for a culture medium formula, identified well, and placed in a designated area for standby application.
2. Culture
2.1 The two 5-ton culturing vats were filled with a total of 10 ton of water.
2.2 14 L of chlorella culture solution was added into the culturing vats and respective amount of ingredients were added according to a ratio of the formula at the same time.
2.2.1 The circulating pump was started and automatic control was performed: after every stop for 2 hours, the circulating pump automatically started to circulate for 0.5 hour, and this step was repeated like this.
2.3 Water in the culturing vats was almost colorless at the beginning, and gradually became light green, until dark green in the culturing process. 5-7 days were required for culture in summer, and 8-15 days were required for culture in winter.
3. Use
3.1 Chlorella fluid was ready for use when the color of the chlorella fluid turned dark green, a valve on a bottom of each culturing vat was opened, and every morning approximate 500 kilograms of chlorella fluid was evenly flowed into 30 10-cube duck pools respectively through pipelines.
3.2 After 500 kilograms of culture solution was used up every day, 500 kilograms of water was supplemented into the culturing vat, and respective amount of ingredients were added according to the ratio of the formula at the same time.
3.3 Chlorella fluid was continuously cultured and used according to the above operating steps.

The growth speed of chlorella was closely related with temperature, light, etc., culture conditions were monitored in real time (mainly by observing and comparing a color of the chlorella fluid every day, and timely sampling to test the concentration in a lab), and the usage amount of approximate 500 kilograms of chlorella fluid was adjusted accordingly, to keep the concentration of the chlorella fluid in the culturing vat above 6*10⁶/ml in real time.

### 2) Water Quality Testing at a Water Inlet I and a Water Outlet O

### Water Quality Testing

### Ammonia Nitrogen

Nanhua Qianmu water quality analysis kit: a test tube was rinsed twice with a water sample first, a water sample was added into the test tube until reaching a scale 2 mL, the test tube was added with ten drops of reagent (A) and ten drops of reagent (B) and shaken up, stood for 10 minutes, and was compared with a standard colorimetric card, and a color code with the same shade indicated the content of ammonia nitrogen (in milligram/liter) in water.

### Nitrite

Nanhua Qianmu water quality analysis kit: a colorimetric tube was rinsed twice with a water sample to be measured, a water sample was added into the colorimetric tube until reaching a scale line, the colorimetric tube was added with 5 drops of nitrite reagent (0), shaken up, and then added with 5 drops of nitrite reagent (1), and after 3 minutes, the colorimetric tube was compared with a color code in this card through visual observation from an opening to a bottom of the tube, and a value above the color code closest to the color in the colorimetric tube was the content of nitrite (as nitrogen, in milligram/liter) in the water sample.

### Dissolved Oxygen

Nanhua Qianmu water quality dissolved oxygen analysis kit: a testing tube was rinsed for two or three times with a water sample to be measured, filled with a water sample, added with 5 drops of reagent dissolved oxygen (I0) and (I1) in turn, immediately capped tightly, and turned upside down, and stood for 3 minutes. The testing tube was uncapped, added with 5 drops of reagent dissolved oxygen (III), capped, turned upside down until a precipitate was completely dissolved (added with reagent dissolved oxygen (III) if the precipitate was not completely dissolved), kept upright, placed to a blank of a colorimetric card, and placed along with the colorimetric card behind an open palm to prevent light. In comparison with a standard colorimetric card, a color code with the same color was the content of dissolved oxygen (in milligram/liter) in the water sample.

### Total Phosphorus

KYORITSU test kit: a pre-embedded thread of a colorimetric tube was pulled out, air was extruded out of the tube, half of water was absorbed into the tube, and the tube was shaken and subject to color comparison within the specified time.

### Cod

LOHAND BIOLOGICAL COD kit: a water sample to be measured was added in a clean colorimetric tube until reaching a 10 mL of scale of the tube, the colorimetric tube was added with a bag of chemical oxygen demand measurement reagent and shaken up for dissolution, and after complete reaction according to reaction time at different temperatures, the colorimetric tube was placed to a blank of a colorimetric card to compare with standard color gradations through visual observation, and a color gradation with the same shade as a solution in the tube was the content of chemical oxygen demand (in mg/L) in water.

### Bod

KYORITSU test kit: a pre-embedded thread of a colorimetric tube was pulled out, air was extruded out of the tube, half of water was absorbed into the tube, and the tube was shaken and subject to color comparison within the specified time.

### 3) Ecological Breeding Chain

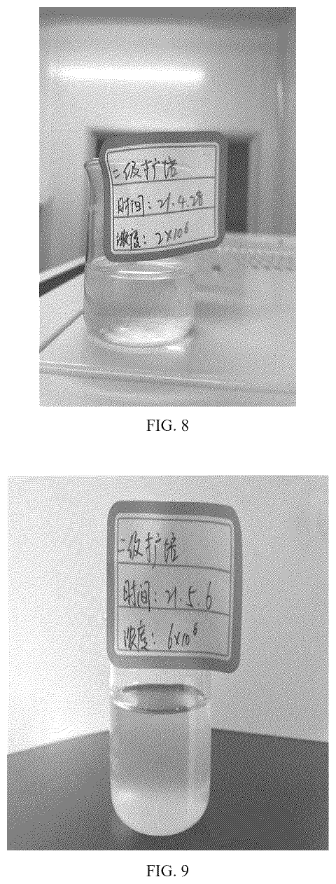

A specific structure is shown as FIG. 8, and includes:

### 3.1 Water Source

Water is stored by building a pool in a river from a mountain, and is channeled into a drinking water area of the poultry farming structure through a 110 water pipe, to ensure that ducks drink pollution-free clean water.

### 3.2. Chlorella

Chlorella in the culture region is conveyed to communicating channels.

### 3.3 Breeding Regions

The breeding regions have a plurality of structures: a small pen B1, a big pool B2, and a big pool B3, as shown in FIG. 12.
(1) The small pen B1: a total of 30 pens exist, and 80-140 laying ducks are raised in each pen. The breeding regions include laying areas B11, activity areas B12, and pools B13. The laying areas are kept dry with husk bedding, the activity areas are hardened with cement, the pools are used for wading of ducks (belonging to wading areas 7), and the pools are partly separated to plant duckweed. Bottoms of the pools are used for breeding catfish, the catfish may consume duck feces, the duckweed may play a role of purifying water (for a specific effect, see water quality test data), and the duckweed growing out every day is used for feeding laying ducks, thereby increasing nutrient and crude fiber for laying ducks, reducing feed cost, and improving the quality of eggs. The activity areas are cleaned with water every day, water in the wading areas is discharged and changed every day, the water discharged enters the first big pool B2, and the laying ducks move in the activity areas at 6-7 o'clock in every morning, and return to the laying areas to rest and lay eggs at 6-7 o'clock in the evening (time may be adjusted according to a seasonal change).
(2) A first big pool B2: 1000-2000 laying ducks are raised in the first big pool, the first big pool includes a laying area B21, an activity area B22, and a wading area B23 (belonging to wading areas 7), half of the wading area is used for wading, and half of the wading area is separated for planting duckweed; catfish is bred in the pool; and water discharged enters a second big pool B3.
(3) A breeding mode of the second big pool B3 is similar to that of the first big pool B2. The second big pool includes a laying area B21, an activity area B22, and a wading area B23 (belonging to wading areas 7). However, different fish is bred in the pools, carp and grass carp are mainly bred in the pool, and water discharged enters a fishpond B4.

It should be noted that the breeding regions may be varied, and not limited to the above settings.

### 3.4 Fishpond B4

Foxtail is planted on a water surface, carp, grass carp, and eel are bred in the fishpond, and if the eel is bred, water quality reaches a standard (for specific data, see a test standard); and tail water of the fishpond enters a settling pond.

### 3.5. Settling Pond B5

The settling pond B5 has a settling effect; tail water of the settling pond B5 is discharged in two ways, the tail water is pumped to a 20-mu orchard B7 in one way, and the tail water is channeled to a greenhouse B6 and used for hydroponic vegetables in the other way; and after reabsorption by root systems of the hydroponic vegetables, water discharged has fully complied with a national irrigation norm.

### Example 2 Results for Primary Culture and Secondary Culture of Chlorella

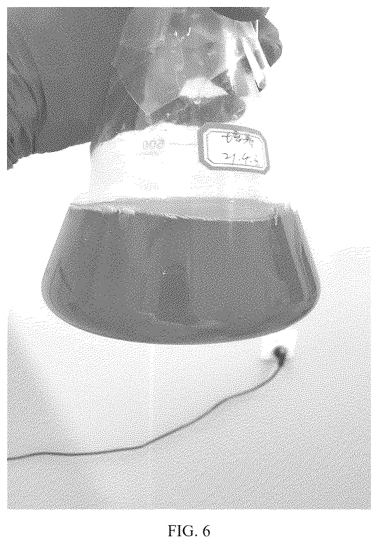

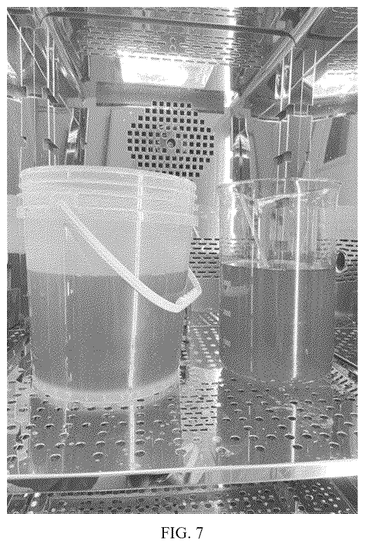

On Apr. 3, 2021, the concentration of chlorella fluid after primary culture of chlorella was tested to be 5.28*10⁶/ml. The chlorella fluid is shown as FIG. 6.

FIG. 7 is a schematic diagram of chlorella fluid after secondary culture of chlorella.

On Apr. 28, 2021, the concentration of chlorella fluid after secondary culture of chlorella was tested to be 2*10⁶/ml. The chlorella fluid is shown as FIG. 8.

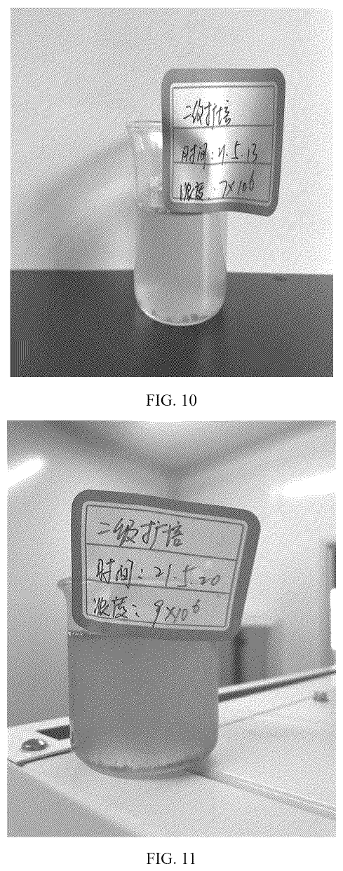

On May 6, 2021, the concentration of chlorella fluid after secondary culture of chlorella was tested to be 6*10⁶/ml. The chlorella fluid is shown as FIG. 9.

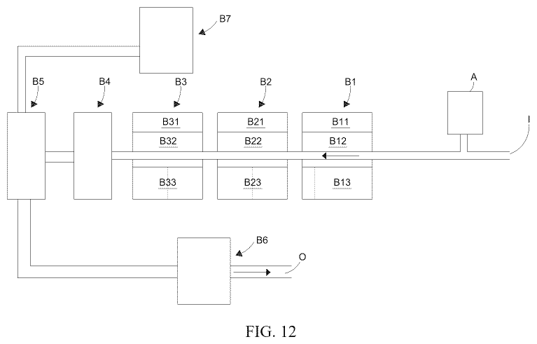

On May 13, 2021, the concentration of chlorella fluid after secondary culture of chlorella was tested to be 7*10⁶/ml. The chlorella fluid is shown as FIG. 10. On May 20, 2021, the concentration of chlorella fluid after secondary culture of chlorella was tested to be 9*10⁶/ml. The chlorella fluid is shown as FIG. 11.

### Example 3 Purification of Combined Chlorella and Duckweed on Water

The influence of different treatment groups on water quality of wading areas was monitored.

TABLE 1
Influence of a treatment group “chlorella + duckweed accounting
for 60% of the area of a duckweed region” on water quality
of wading areas (all water quality tests were recorded in 2021)

| Test date | Jul. 26 | Jul. 27 | Aug. 16 | Aug. 17 | Aug. 18 | Aug. 19 |
|---|---|---|---|---|---|---|
| pH (7.5-8.4) | 7.6 | 7.4 | 7.6 | 7.4 | 7.0 | 7.4 |
| Ammonia | 0.7 | 0.4 | 0.2 | 0.2 | 0 | 0 |
| nitrogen | | | | | | |
| (≤0.5) | | | | | | |
| Nitrite (≤0.1) | 0.005 | 0.005 | 0.005 | 0.002 | 0.005 | 0.005 |
| Dissolved | 6 | 7 | 4 | 4.0 | 6.0 | 7.0 |
| oxygen (3-8) | | | | | | |
| Total | 10 | 8 | 5 | 5 | 3 | 5 |
| phosphorus | | | | | | |
| (≤8.0) | | | | | | |
| Cod ≤400 | 150 | 40 | 30 | 30 | 10 | 10 |
| Bod ≤150 | 150 | 90 | 50 | 50 | 100 | 50 |
| Water | 31.6 | 28.6 | 27.8 | 27.8 | 24.9 | 25.1 |
| temperature | | | | | | |
| (3° C.-38° C.) | | | | | | |
| Remarks | Tested at | Tested at | Tested at | Tested at | Tested at | Tested at |
| | half past | half past | half past | half past | nine in the | nine in |
| | three in the | nine in the | twelve at | four in the | morning, | the |
| | afternoon, | morning, | noon, inlet | afternoon, | and ducks | morning, |
| | and water | water for | and outlet | and not | have been | and |
| | not | duckweed | flow | cleaned for | out for | ducks |
| | changed | not changed, | increased | nearly half | about two | were |
| | for five | and water in | by three | a month | hours | released |
| | days | the activity | times, and | | | after a |
| | | area for | duck sheds | | | water |
| | | ducks | not cleaned | | | sample |
| | | changed | recently. | | | was |
| | | | Water | | | taken |
| | | | quality got | | | |
| | | | better | | | |
| | | | apparently. | | | |

TABLE 2
Influence of a treatment group “chlorella” on water quality
of wading areas (all water quality tests were recorded in 2021)

| Test date | Jul. 26 | Jul. 27 | Aug. 18 | Aug. 19 |
|---|---|---|---|---|
| pH (7.5-8.4) | 6.6 | 6.5 | 7.4 | 7.0 |
| Ammonia nitrogen | 0.4 | 0.1 | 0 | 0 |
| (≤0.5) | | | | |
| Nitrite (≤0.1) | 0.005 | 0.005 | 0.008 | 0.008 |
| Dissolved oxygen | 6 | 7 | 6.0 | 6.0 |
| (3-8) | | | | |
| Total phosphorus | 5 | 3 | 2 | 3 |
| (≤8.0) | | | | |
| Cod ≤400 | 30 | 30 | 10 | 10 |
| Bod ≤150 | 100 | 80 | 10 | 10 |
| Water temperature | 31.3 | 27.3 | 25.7 | 26.6 |
| (3 °C-38 °C) | | | | |
| Remarks | Tested at half | Tested at half | Tested at nine | Tested at nine |
| | past three in | past nine in | in the morning, | in the morning, |
| | the afternoon, | the morning | and water | and duck sheds |
| | and water changed | | changed | cleaned and |
| | every two or | | yesterday | water changed |
| | three days | | | every day |

TABLE 3
Influence of a treatment group “duckweed accounting for 60% of the
area of a duckweed region” on water quality of wading areas

| | Date | | |
|---|---|---|---|
| | Aug. 17, | Aug. 18, | Aug. 19, |
| | 2021 | 2021 | 2021 |
| pH (7.5-8.4) | 7.4 | 7.4 | 7.4 |
| Ammonia nitrogen | 0 | 0 | 0 |
| (≤0.5) | | | |
| Nitrite (≤0.1) | 0.01 | 0.008 | 0.008 |
| Dissolved oxygen | 3.0 | 8.0 | 7.0 |
| (3-8) | | | |
| Total phosphorus | 3 | 1 | 1 |
| (≤8.0) | | | |
| Cod ≤400 | 20 | 10 | 10 |
| Bod ≤150 | 30 | 80 | 10 |
| Water temperature | 28.4 | 25.2 | 25.2 |
| (3 °C-38 °C) | | | |
| Remarks | Water not | Tested at nine | Tested at nine |
| | changed for | in the morning, | in the morning, |
| | three days | water not changed | and ducks were |
| | | recently, and ducks | released after |
| | | have been out for | a water sample |
| | | about two hours | was taken |

### Example 4 Purification of the Ecological Breeding Chain on Water at the Water Inlet I and the Water Outlet O

Water quality at the water inlet I and the water outlet O was continuously monitored, and results were shown as Table 4 and Table 5.

TABLE 4a
2021-2022 records of water quality at the water inlet

| | Water quality at the water inlet (mg/L) | | | | |
|---|---|---|---|---|---|
| | | Ammonia | | Dissolved | Water |
| | pH | nitrogen | Nitrite | oxygen | temperature |
| Date | (7.5-8.4) | (≤0.5) | (≤0.1) | (3-8) | (3 °C-38 °C) |
| 12.31 | 8.2 | 0.2 | 0.05 | 3.0 | 13.4 |
| 1.7 | 8.2 | 0.4 | 0.05 | 3.0 | 15.5 |
| 1.14 | 6.6 | 0.4 | 0.05 | 3.0 | 13.6 |
| 1.21 | 6.6 | 0.2 | 0.02 | 3.0 | 18.9 |
| 1.28 | 6.6 | 0.2 | 0.01 | 3.0 | 16.8 |
| 2.4 | 6.6 | 0.2 | 0.01 | 3.0 | 9.5 |
| 2.11 | 6.6 | 0.2 | 0.01 | 3.0 | 14.8 |
| 2.18 | 7.0 | 0.2 | 0.05 | 3.0 | 13.7 |
| 2.25 | 7.0 | 0.2 | 0.05 | 3.0 | 13.2 |
| 3.4 | 7.0 | 0.2 | 0.05 | 3.0 | 20.7 |
| 3.11 | 7.0 | 0.2 | 0.05 | 3.0 | 20.2 |
| 3.18 | 7.4 | 0 | 0.01 | 3.0 | 22.3 |
| 3.25 | 7.4 | 0 | 0.01 | 3.0 | 19.4 |
| 4.1 | 7.4 | 0 | 0.01 | 3.0 | 15.8 |
| 4.8 | 6.4 | 0 | 0 | 4.0 | 17.2 |
| 4.15 | 6.4 | 0 | 0 | 4.0 | 20.1 |
| 4.22 | 6.6 | 0.2 | 0.005 | 6.0 | 22.1 |
| 4.29 | 6.6 | 0 | 0.005 | 5.0 | 22.4 |
| 5.6 | 6.6 | 0 | 0.005 | 6.0 | 19.6 |

TABLE 4b
2021-2022 records of water quality at the water inlet

Water quality at the water inlet (mg/L)

| | pH | Ammonia | Nitrite | Dissolved |
|---|---|---|---|---|
| Date | (7.5-8.4) | nitrogen (≤0.5) | (≤0.1) | oxygen (3-8) |
| 5.13 | 7.2 | 0 | 0.005 | 7.0 |
| 5.14 | | | | |
| 6.3 | 6.6 | 0 | 0.01 | 7.0 |
| 6.24 | 7.0 | 0 | 0.01 | 7.0 |
| 7.1 | 7.0 | 0.1 | 0.01 | 8.0 |
| 7.8 | 6.6 | 0 | 0.01 | 7.0 |
| 7.15 | 6.6 | 0 | 0.01 | 7.0 |
| 7.22 | 6.6 | 0 | 0.005 | 8.0 |
| 7.29 | 6.6 | 0 | 0.005 | 7.0 |
| 8.5 | 6.6 | 0 | 0.005 | 7.0 |
| 8.12 | 6.6 | 0 | 0.005 | 7.0 |
| 8.27 | 6.4 | 0.4 | 0.05 | 6.0 |
| 9.2 | 6.4 | 0.3 | 0.05 | 7.0 |
| 9.9 | 6.6 | 0.4 | 0.01 | 6.0 |
| 9.16 | 7.4 | 0.5 | 0.01 | 7.0 |
| 9.23 | 7.4 | 0.5 | 0.01 | 7.0 |
| 9.30 | 7.6 | 0.4 | 0.01 | 5.0 |
| 10.7 | 7.6 | 0.4 | 0.01 | 7.0 |
| 10.14 | 7.6 | 0.5 | 0.01 | 7.0 |
| 10.21 | 7.6 | 0.5 | 0.01 | 7.0 |
| 10.28 | 7.6 | 0.5 | 0.01 | 7.0 |
| 11.4 | 7.4 | 0.4 | 0.01 | 7.0 |
| 11.12 | 7.4 | 0.4 | 0.01 | 7.0 |
| 11.19 | 7.4 | 0.4 | 0.01 | 8.0 |
| 11.26 | 7.2 | 0 | 0 | 8.0 |
| 12.3 | 7.6 | 0 | 0 | 8.0 |

TABLE 4c
2021-2022 records of water quality at the water inlet

| | Water quality at the water inlet (mg/L) | | | |
|---|---|---|---|---|
| | Total | | | Water temperature |
| Date | phosphorus (≤8.0) | Cod ≤400 | Bod ≤150 | (3 °C-38 °C) |
| 5.13 | 0 | 0 | 80 | 20.6 |
| 5.14 | 0 | 15 | 20 | |
| 6.3 | 0 | 15 | 10 | 25.3 |
| 6.24 | 0 | 25 | 10 | 24.5 |
| 7.1 | 0 | 15 | 10 | 25.3 |
| 7.8 | 0 | 10 | 0 | 23.6 |
| 7.15 | 0 | 10 | 0 | 31.1 |
| 7.22 | 0 | 10 | 0 | 34.1 |
| 7.29 | 0 | 10 | 0 | 31.5 |
| 8.5 | 0 | 10 | 0 | 25.5 |
| 8.12 | 0 | 10 | 0 | 27.1 |
| 8.27 | 3 | 10 | 0 | 25.1 |
| 9.2 | 3 | 10 | 0 | 25.7 |
| 9.9 | 3 | 10 | 0 | 25.2 |
| 9.16 | 3 | 10 | 2 | 24.7 |
| 9.23 | 3 | 10 | 10 | 22.1 |
| 9.30 | 3 | 10 | 10 | 23.8 |
| 10.7 | 0 | 10 | 10 | 22.1 |
| 10.14 | 0 | 10 | 10 | 19.5 |
| 10.21 | 0 | 10 | 10 | 18.2 |
| 10.28 | 0 | 10 | 10 | 21.2 |
| 11.4 | 0 | 10 | 10 | 19.5 |
| 11.12 | 0 | 10 | 10 | 19 |
| 11.19 | 0 | 10 | 10 | 20.4 |
| 11.26 | 0 | 0 | 0 | 19.6 |
| 12.3 | 0 | 10 | 10 | 14.4 |

TABLE 5
2021-2022 records of water quality at the water outlet

| | Water quality at the water outlet (mg/L) | | | | |
|---|---|---|---|---|---|
| | | Ammonia | | Dissolved | Water |
| | pH | nitrogen | Nitrite | oxygen | temperature |
| Date | (6.0-9.0) | (0.02-150) | (≤15) | (≥3.0) | ( ) |
| 12.31 | 8.4 | 0.5 | 0.05 | 3.0 | 12.2 |
| 1.7 | 8.2 | 0.7 | 0.1 | 3.0 | 14.9 |
| 1.14 | 6.4 | 0.5 | 0.05 | 3.0 | 12.2 |
| 1.21 | 7.6 | 1.0 | 0.05 | 3.0 | 14.9 |
| 1.28 | 7.6 | 1.0 | 0.1 | 3.0 | 16.8 |
| 2.4 | 7.6 | 1.0 | 0.1 | 3.0 | 10.4 |
| 2.11 | 7.6 | 1.0 | 0.1 | 3.0 | 11.8 |
| 2.18 | 7.6 | 1.0 | 0.1 | 3.0 | 14.3 |
| 2.25 | 7.6 | 1.0 | 0.1 | 3.0 | 11.2 |
| 3.4 | 7.6 | 1.0 | 0.1 | 3.0 | 19.9 |
| 3.11 | 7.6 | 0.6 | 0.1 | 3.0 | 18.3 |
| 3.18 | 7.4 | 0.6 | 0.05 | 2.0 | 21.2 |
| 3.25 | 7.4 | 0.6 | 0.05 | 2.0 | 18.1 |
| 4.1 | 7.4 | 0.6 | 0.05 | 2.0 | 19.1 |
| 4.8 | 7.4 | 0.2 | 0.005 | 4.0 | 19.1 |
| 4.15 | 7.4 | 0.2 | 0.005 | 4.0 | 21.3 |
| 4.22 | 7.4 | 0.2 | 0.01 | 3.0 | 19.2 |
| 4.29 | 7.6 | 0.2 | 0.01 | 7.0 | 24.9 |
| 5.6 | 7.6 | 0 | 0.005 | 7.0 | 21.5 |
| 5.13 | 7.6 | 0 | 0.005 | 4.0 | 22.5 |
| 5.14 | | | | | |
| 6.3 | 7.6 | 0.2 | 0.005 | 6.0 | 25.7 |
| 6.24 | 7.5 | 0.3 | 0.03 | 7.0 | 26.9 |
| 7.1 | 7.6 | 0.7 | 0.03 | 7.0 | 29.2 |
| 7.8 | 7.6 | 0.5 | 0.05 | 7.0 | 26.8 |
| 7.15 | 7.6 | 0.4 | 0.05 | 7.0 | 30.1 |
| 7.22 | 7.6 | 0.4 | 0.1 | 4.0 | 31.8 |
| 7.29 | 7.6 | 0.4 | 0.05 | 7.0 | 32 |
| 8.5 | 7.6 | 0.3 | 0.01 | 6.0 | 28.1 |
| 8.12 | 7.6 | 0.4 | 0.05 | 7.0 | 29.4 |
| 8.15 | 7.6 | 0.4 | 0.08 | 6.0 | 29.9 |
| 8.17 | 7.6 | 0.1 | 0.05 | 7.0 | 30.8 |
| 8.19 | 7.6 | 0.2 | 0.05 | 7.0 | 28.2 |
| 8.27 | 8.3 | 1.2 | 0.01 | 7.0 | 28.2 |
| 9.2 | 7.6 | 0.8 | 0.01 | 7.0 | 26.9 |
| 9.9 | 7.6 | 1.1 | 0.01 | 6.0 | 25.9 |
| 9.16 | 7.6 | 1.1 | 0.08 | 6.0 | 24.7 |
| 9.23 | 7.6 | 1.1 | 0.08 | 6.0 | 25 |
| 9.30 | 7.6 | 1.1 | 0.05 | 6.0 | 25.9 |
| 10.7 | 7.6 | 1.1 | 0.05 | 6.0 | 25.6 |
| 10.14 | 7.6 | 1.1 | 0.05 | 6.0 | 20.9 |
| 10.21 | 7.6 | 1.2 | 0.05 | 7.0 | 19.6 |
| 10.28 | 7.6 | 1.2 | 0.05 | 7.0 | 20.5 |
| 11.4 | 7.6 | 1.1 | 0.05 | 7.0 | 19.4 |
| 11.12 | 7.6 | 1.1 | 0.05 | 6.0 | 20.8 |
| 11.19 | 7.6 | 1.2 | 0.05 | 6.0 | 20.9 |
| 11.26 | 7.4 | 1.5* | 0.12 | 6.0 | 20.2 |
| 12.3 | 7.4 | 1.4 | 0.15 | 6.0 | 14.4 |

| | Total | | | |
|---|---|---|---|---|
| | phosphorus | | | |
| | (≤8.0), | cod ≤400, | Bod ≤150, | |
| | reagent | reagent | reagent | |
| | standard | standard | standard | |
| | 0-100 | 0-250 | 0-500 | Remarks |
| 5.13 | 5 | 0 | 0 | |
| 5.14 | 0 | 15 | 100 | A water sample |
| | | | | diluted by ten times |
| 6.3 | 0 | 20 | 40 | |
| 6.24 | 5 | 20 | 20 | |
| 7.1 | 7 | 20 | 50 | |
| 7.8 | 6 | 20 | 40 | Water quality tested in |
| | | | | recent two weeks was |
| | | | | poor compared with |
| | | | | previous data, |
| | | | | because inlet water |
| | | | | was decreased |
| | | | | recently, an activity |
| | | | | range of ducks was |
| | | | | expanded, and the |
| | | | | previous settling |
| | | | | range was reduced |
| | | | | due to proximity to |
| | | | | the water outlet. |
| 7.15 | 7 | 20 | 30 | |
| 7.22 | 6 | 25 | 50 | |
| 7.29 | 5 | 30 | 50 | |
| 8.5 | 6 | 30 | 60 | The water outlet was |
| | | | | provided with a buoy, |
| | | | | and water quality was |
| | | | | slightly better. |
| 8.12 | 3 | 30 | 50 | |
| 8.15 | 5 | 30 | 60 | A drain pipe of a pond |
| | | | | 2 was broken, and |
| | | | | water did not enter a |
| | | | | pond 3 overnight. |
| 8.17 | 6 | 20 | 80 | |
| 8.19 | 2 | 25 | 10 | |
| 8.27 | 7 | 25 | 30 | |
| 9.2 | 7 | 25 | 30 | |
| 9.9 | 7 | 50 | 30 | |
| 9.16 | 6 | 20 | 30 | |
| 9.23 | 8 | 20 | 30 | |
| 9.30 | 6 | 20 | 30 | |
| 10.7 | 6 | 20 | 30 | |
| 10.14 | 5 | 20 | 30 | |
| 10.21 | 7 | 20 | 30 | |
| 10.28 | 5 | 20 | 30 | |
| 11.4 | 5 | 20 | 30 | |
| 11.12 | 6 | 10 | 10 | |
| 11.19 | 7 | 10 | 10 | |
| 11.26 | 5 | 10 | 10 | *: (new reagent 1.7) |
| | | | | has reached the |
| | | | | highest level of the |
| | | | | colorimetric card |
| 12.3 | 5 | 10 | 10 | |

### Example 5 Purification of the Ecological Breeding Chain on Water at the First Big Pool and the Second Big Pool

Water quality in the first big pool and the second big pool was continuously monitored, and results were shown as Table 6 and Table 7.

TABLE 6a
Water quality in the first big pool

| | Water quality (mg/L) | | | | |
|---|---|---|---|---|---|
| | | Ammonia | | Dissolved | Total |
| Date | pH | nitrogen | Nitrite | oxygen | nitrogen |
| 2023 Aug. 4 | 7 | 0.4 | 0 | 1 | 0 |
| morning | | | | | |
| Afternoon | 7 | 0.4 | 0 | 1 | 0 |
| 2023 Aug. 5 | 7 | 0.2 | 0 | 4 | 0 |
| morning | | | | | |
| Afternoon | 7.2 | 0.4 | 0 | 1 | 0 |
| 2023 Aug. 6 | 6.8 | 0.4 | 0 | 1 | 0 |
| morning | | | | | |
| Afternoon | 7 | 0.4 | 0 | 1 | 0 |
| Mean | 7 | 0.37 | 0 | 1.5 | 0 |

TABLE 6b
Water quality in the first big pool

| | Water quality (mg/L) | | | |
|---|---|---|---|---|
| | Ammonia | | | Water |
| | nitrogen | | | temperature |
| Date | (fresh water) | Cod ≤400 | Bod ≤150 | (3 °C-38 °C) |
| 2023 Aug. 4 | 0.2 | 30 | 20 | 26.1 |
| morning | | | | |
| Afternoon | 0.2 | 30 | 20 | 29.1 |
| 2023 Aug. 5 | 0.2 | 30 | 20 | 25.8 |
| morning | | | | |
| Afternoon | 0.2 | 30 | 20 | 28.8 |
| 2023 Aug. 6 | 0.2 | 30 | 0 | 25.8 |
| morning | | | | |
| Afternoon | 0.2 | 30 | 40 | 29.1 |
| Mean | 0.2 | 30 | 20 | 27.45 |

TABLE 7a
Water quality in the second big pool

| | Water quality (mg/L) | | | | |
|---|---|---|---|---|---|
| | | Ammonia | | Dissolved | Total |
| Date | pH | nitrogen | Nitrite | oxygen | nitrogen |
| 2023 Aug. 4 | 6.6 | 0.6 | 0 | 1 | 10 |
| morning | | | | | |
| Afternoon | 7.4 | 0.6 | 0 | 2 | 10 |
| 2023 Aug. 5 | 7 | 0.6 | 0 | 3 | 10 |
| morning | | | | | |
| Afternoon | 7.4 | 0.6 | 0 | 5 | 5 |
| 2023 Aug. 6 | 6.8 | 0.6 | 0 | 4 | 5 |
| morning | | | | | |
| Afternoon | 7.4 | 0.6 | 0 | 7 | 5 |
| Mean | 7.1 | 0.6 | 0 | 3.6667 | 7.5 |

TABLE 7b
Water quality in the second big pool

| | Water quality (mg/L) | | | |
|---|---|---|---|---|
| | Ammonia | | | |
| | nitrogen | | | Water temperature |
| Date | (fresh water) | Cod ≤400 | Bod ≤150 | (3 °C-38 °C) |
| 2023 Aug. 4 | 1.4 | 30 | 20 | 27.9 |
| morning | | | | |
| Afternoon | 1.4 | 30 | 20 | 30.4 |
| 2023 Aug. 5 | 1.4 | 30 | 40 | 28.5 |
| morning | | | | |
| Afternoon | 1.7 | 30 | 40 | 30.1 |
| 2023 Aug. 6 | 1.4 | 30 | 20 | 28.3 |
| morning | | | | |
| Afternoon | 1.4 | 30 | 20 | 30.7 |
| Mean | 1.45 | 30 | 26.66667 | 29.31666667 |

1. A poultry farming structure, comprising a culture region and breeding regions, wherein a housing of the culture region is transparent or semitransparent, so that light irradiates into the culture region from outside or the culture region is in communication with the outside, and the culture region is in communication with the breeding regions.

2. The poultry farming structure according to claim 1, comprising a plurality of compartments arranged from top to bottom in sequence, wherein each of the plurality of compartments comprises a plurality of the breeding regions, and the culture region is in communication with the breeding regions of each of the plurality of compartments through a plurality of communicating channels.

3. The poultry farming structure according to any one of claim 1, wherein the breeding regions are provided with wading areas, and each of the plurality of communicating channels is connected to the wading areas in the breeding regions.

4. The poultry farming structure according to claim 3, wherein parts of the plurality of communicating channels located in the breeding regions are provided with openings.

5. The poultry farming structure according to claim 4, wherein the parts of the plurality of communicating channels located in the breeding regions are flush with bearing surfaces of the breeding regions or the parts of the plurality of communicating channels located in the breeding regions are 0-50 cm higher than the bearing surfaces of the breeding regions, and the openings are located at tops of the plurality of communicating channels, wherein the bearing surfaces are surfaces for bearing poultry.

6. The poultry farming structure according to claim 4, wherein the parts of the plurality of communicating channels located in the breeding regions are more than 100 cm higher than bearing surfaces of the breeding regions, and bottoms of the plurality of communicating channels are provided with spray openings, wherein the bearing surfaces are surfaces for bearing poultry.

7. The poultry farming structure according to claim 2, wherein a valve is arranged between the plurality of communicating channels and the culture region.

8. The poultry farming structure according to claim 1, wherein the culture region is located at a top of the poultry farming structure.

9. The poultry farming structure according to claim 1, further comprising a liquid pump, wherein the liquid pump is configured to convey liquid to the culture region.

10. A poultry farming method being applied to the poultry farming structure according to claim 1, comprising: allowing poultry to ingest chlorella and duckweed.

11.-13. (canceled)

14. The poultry farming method according to claim 10, wherein the poultry ingest the chlorella and the duckweed simultaneously or alternately.

15. The poultry farming method according to claim 14, wherein the chlorella and the duckweed are provided in a form of a single component, and not provided simultaneously in timing.

16. The poultry farming method according to claim 10, comprising adding the chlorella into drinking water for the poultry so that the chlorella is ingested by the poultry, wherein a concentration of the chlorella in the drinking water for the poultry is 6*10⁶/mL, 7*10⁶/mL, 8*10⁶/mL, 9*10⁶/mL, 10*10⁶/mL, 11*10⁶/mL, 12*10⁶/mL, 13*10⁶/mL, 14*10⁶/mL, 15*10⁶/mL, 16*10⁶/mL, 17*10⁶/mL, 18*10⁶/mL, 19*10⁶/mL, 20*10⁶/mL, or more than 20*10⁶/mL.

17. The poultry farming method according to claim 10, comprising culturing the duckweed in wading areas for the poultry, wherein the poultry ingests the duckweed in the wading areas.

18. A method for purifying aquaculture water of the poultry farming structure according to claim 1, comprising conveying chlorella to the aquaculture water, and culturing duckweed on a surface of the aquaculture water at a same time.

19. The method for purifying aquaculture water of claim 18, wherein a concentration of the chlorella conveyed to the aquaculture water is 6*10⁶/mL, 7*10⁶/mL, 8*10⁶/mL, 9*10⁶/mL, 10*10⁶/mL, 11*10⁶/mL, 12*10⁶/mL, 13*10⁶/mL, 14*10⁶/mL, 15*10⁶/mL, 16*10⁶/mL, 17*10⁶/mL, 18*10⁶/mL, 19*10⁶/mL, 20*10⁶/mL, or more than 20*10⁶/mL.

20. The method for purifying aquaculture water of claim 18, wherein the duckweed accounts for at least 60% of the surface of the aquaculture water.

21. A three-dimensional ecological breeding method, being applied to the poultry farming structure according to claim 3, and comprising:
adding chlorella to the culture region;
culturing duckweed in the wading areas; and
conveying a liquid containing the chlorella to the wading areas through the plurality of communicating channels.

22. The three-dimensional ecological breeding method of claim 21, wherein a concentration of the chlorella in the wading areas is 6*10⁶/mL, 7*10⁶/mL, 8*10⁶/mL, 9*10⁶/mL, 10*10⁶/mL, 11*10⁶/mL, 12*10⁶/mL, 13*10⁶/mL, 14*10⁶/mL, 15*10⁶/mL, 16*10⁶/mL, 17*10⁶/mL, 18*10⁶/mL, 19*10⁶/mL, 20*10⁶/mL, or more than 20*10⁶/mL.

23. The three-dimensional ecological breeding method of claim 21, wherein water in the wading areas is discharged into a fishpond, water in the fishpond is discharged into a settling pond, and water in the settling pond is discharged into a greenhouse and an orchard.
